(19) United States
(12) Patent Application Publication
(10) Pub. No.: US 2015/0272879 A1
(43) Pub. Date: Oct. 1, 2015

(54) ESSENTIAL OIL AND HERBAL EXTRACT MIST COMPOSITION AND METHOD OF DELIVERY

(71) Applicants: Yervand Simonyan, St. Louis, MO (US); Anahit Simonyan, St. Louis, MO (US)

(72) Inventors: Yervand Simonyan, St. Louis, MO (US); Anahit Simonyan, St. Louis, MO (US)

(21) Appl. No.: 14/336,315

(22) Filed: Jul. 21, 2014

Related U.S. Application Data
(60) Provisional application No. 61/972,064, filed on Mar. 28, 2014.

(51) Int. Cl. A61K 9/00; A61K 36/00; A61M 15/00; A61K 47/10; A61M 11/00; A61K 31/21; A61K 47/34

(52) U.S. Cl. CPC A61K 9/0078; A61K 31/21; A61K 36/00; A61K 47/34; A61K 47/10; A61M 11/00; A61M 15/0021

(57) ABSTRACT

A liquid composition that includes essential oils, herbal extracts or a combination thereof is provided. The liquid composition further includes a base carrier. The base carrier may include propylene glycol, vegetable glycerin, water or a combination thereof, or any other substance with suitable physical and chemical properties to dilute essential oils/herbal extracts while also being safe for inhalation. The liquid composition may be converted by an atomizer into a mist and then inhaled by a user.

### CROSS-REFERENCE TO RELATED APPLICATION

This application claims the benefit of priority of U.S. provisional application No. 61/972,064, filed Mar. 28, 2014, the contents of which are herein incorporated by reference.

### BACKGROUND OF THE INVENTION

The present invention relates to essential oil and/or herbal extract delivery and, more particularly, to a mist composition containing essential oils and/or herbal extracts.

Aromatherapy is a form of alternative medicine that uses volatile plant materials, known as essential oils, herbal extracts, and other aromatic compounds for the purpose of altering a person's mind, mood, cognitive function or health.

Current methods of delivery for administration of essential oils such as vapor diffusers, candle fumes, steam evaporators, and topical applications, do not provide for standardized dosing and/or are expensive, cumbersome, and difficult to use. The current methods of delivery require specialized knowledge and experience for safe and effective use. These barriers greatly restrict use of aromatherapy from a broader population which could otherwise benefit from such use. Further, existing methods have less predictable outcomes due to the non-standardized dosing preparation and slow, insufficient, and/or erratic absorption.

As can be seen, there is a need for an improved delivery of essential oils and herbal extracts.

### SUMMARY OF THE INVENTION

In one aspect of the present invention, a composition comprises an essential oil, an herbal extract or a combination thereof, wherein the composition further comprises a base carrier comprising propylene glycol, vegetable glycerin, water, or a combination thereof.

In another aspect of the present invention, a method of delivery comprises: providing a liquid composition comprising an essential oil, an herbal extract or a combination thereof, converting the liquid composition to a mist composition using an atomizer; and inhaling the mist composition.

These and other features, aspects and advantages of the present invention will become better understood with reference to the following drawings, description and claims.

### BRIEF DESCRIPTION OF THE DRAWINGS

FIG. 1 is a side elevation view, with parts broken away, of the present invention in use; and

FIG. 2 is a flow chart of an embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The present invention provides the physical and chemical properties to dilute essential oil(s)/herbal extract(s) in base carrier solution, atomize this solution into a safe and breathable mist, and deliver this mist into the respiratory tract for absorption in the lungs of the user. The present invention includes a method of delivery for aromatherapy via direct oral inhalation of mist containing essential oil(s)/herbal extract(s). The present invention allows for pre-determined, standardized dosing of essential oil(s) and/or related herbal extract(s) administered through direct oral inhalation in the form of a mist that contains the essential oil(s)/herbal extract(s). By standardizing dosage of essential oils/herbal extracts (to the established safe concentration, generally accepted to be up to about 5%) for direct oral inhalation, the desired effect(s) are more easily obtained with less chance for dosing errors and adverse effects.

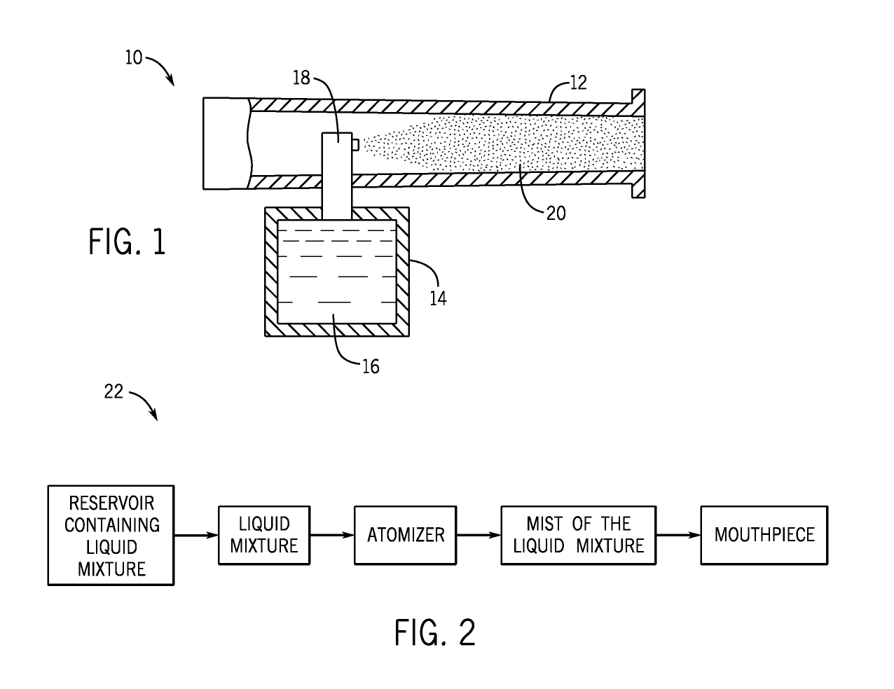

Referring to FIGS. 1 and 2, the present invention includes a liquid composition 16 that includes essential oils, herbal extracts or a combination thereof. The liquid composition 16 further includes a base carrier. The base carrier may include propylene glycol, vegetable glycerin, water or a combination thereof, or any other substance with suitable physical and chemical properties to dilute essential oils/herbal extracts while also being safe for inhalation. Different combinations of herbs and oils may be used for different purposes, for example relaxation, digestive aid, sleeps enhancement, energy stimulant, and the like.

In certain embodiments, the base carrier may include 40% propylene glycol and 60% vegetable glycerin. The base carrier mixture may then be used to dissolve the essential oil(s)/herbal extract(s) to form the composition 16. The composition 16 may contain up to about 5% essential oil(s)/herbal extract(s). In alternate embodiments, the proportions of propylene glycol and vegetable glycerin may be modified, and may include the addition of water.

The liquid composition 16 may be converted into a mist composition 20 for inhalation by a user. The liquid composition 20 may be retained within a reservoir 14. An atomizer 18 may be activated to convert the liquid composition 20 in the reservoir 14 into the mist composition 20. The atomizer 18 may be either mechanical, electrical, or of any other type. The mist composition 20 may then be inhaled from a mouthpiece 12.

The present invention facilitates the direct oral inhalation of a mist containing essential oil(s)/herbal extract(s). The mist is created from atomizing a liquid composition of base carrier and essential oil(s)/herbal extract(s) to a concentration of up to about 5%. The base carrier can be any combination of: propylene glycol, vegetable glycerin, water, and/or any other substance with suitable physical and chemical properties to dilute essential oils/herbal extracts while also being safe for inhalation. The liquid mixture of base carrier and essential oil(s)/herbal extract(s) may or may not contain flavorings and/or other additives.

By direct oral inhalation of mist containing essential oil(s)/herbal extract(s), the therapeutic and/or recreational effects of these oils/herbal extracts can be attained in a way which allows for standardized dosing, increased absorption, shorter time for onset of effects, and a generally new and superior experience of aromatherapy. These innovations may significantly reduce barriers which greatly restrict use of aromatherapy from a broader population which could otherwise benefit from such use.

It should be understood, of course, that the foregoing relates to exemplary embodiments of the invention and that modifications may be made without departing from the spirit and scope of the invention as set forth in the following claims.

What is claimed is:

1. A composition comprising an essential oil, an herbal extract or a combination thereof, wherein the composition further comprises a base carrier comprising propylene glycol, vegetable glycerin, water, or a combination thereof.

2. The composition of claim 1, wherein the essential oil, the herbal extract, or the combination thereof comprises up to about 5% of the composition.

3. A method of delivery comprising:
providing a liquid composition comprising an essential oil, an herbal extract or a combination thereof;
converting the liquid composition to a mist composition using an atomizer; and
inhaling the mist composition.

4. The method of claim 2, wherein the liquid composition further comprises a base carrier comprising propylene glycol, vegetable glycerin, water or a combination thereof.

5. The method of claim 2, wherein the mist composition is delivered through a mouthpiece.

6. The method of claim 3, wherein the liquid composition is contained within a reservoir.
